SEQUENCE LISTING (5 sequences)

SEQ ID NO: 1 - protein, 475 aa, synthetic construct
  source 1..475: note = inorganic ATP sulfurylase
MNTALAQQIANEGGVEAWMIAQQHKSLLRFLTCGSVDDGKSTLIGRLLHDTRQIYEDQLSSLHNDSKRHGTQGEKLDLALLVDGLQAEREQGITIDVAYRYFSTEKRKFIIADTPGHEQYTRNMATGASTCELAILLIDARKGVLDQTRRHSFISTLLGIKHLVVAINKMDLVDYSEETFTRIRKDYLTFAEQLPGNLDIRFVPLSALEGDNVASQSESMPWYSGPTLLEVLETVEIQRVVDAQPMRFPVQYVNRPNLDFRGYAGTLASGRVEVGQRVKVLPSGVESNVARIVTFDGDREEAFAGEAITLVLTDEIDISRGDLLLAADEALPAVQSASVDVVWMAEQPLSPGQSYDIKIAGKKTRARVDGIRYQVDINNLTQREVENLPLNGIGLVDLTFDEPLVLDRYQQNPVTGGLIFIDRLSNVTVGAGMVHEPVSQATAAPSEFSAFELELNALVRRHFPHWGARDLLGDK

SEQ ID NO: 2 - DNA, 29 bp, synthetic construct
  source 1..29: mol_type = other DNA; note = P5
aatgatacggcgaccaccgagatctacac

SEQ ID NO: 3 - DNA, 24 bp, synthetic construct
  source 1..24: mol_type = other DNA; note = P7
caagcagaagacggcatacgagat

SEQ ID NO: 4 - DNA, 29 bp, synthetic construct
  source 1..29: mol_type = other DNA; note = P5 prime
gtgtagatctcggtggtcgccgtatcatt

SEQ ID NO: 5 - DNA, 24 bp, synthetic construct
  source 1..24: mol_type = other DNA; note = P7 prime
atctcgtatgccgtcttctgcttg

(19) United States
(12) Patent Application Publication
(10) Pub. No.: US 2024/0124929 A1
(43) Pub. Date: Apr. 18, 2024

(54) MESOPHILIC COMPOSITIONS FOR NUCLEIC ACID AMPLIFICATION

(71) Applicant: Illumina, Inc., San Diego, CA (US)

(72) Inventor: Justin Robbins, San Diego, CA (US)

(73) Assignee: Illumina, Inc., San Diego, CA (US)

(21) Appl. No.: 18/476,031

(22) Filed: Sep. 27, 2023

Related U.S. Application Data
(60) Provisional application No. 63/411,965, filed on Sep. 30, 2022.

(51) Int. Cl. C12Q 1/6869; C12Q 1/6844

(52) U.S. Cl. CPC C12Q 1/6869; C12Q 1/6844; C12Q 1/6806

(57) ABSTRACT

This disclosure relates to novel amplification compositions and methods, in particular for use in nucleic acid amplification and sequencing, preferably that do not involve reagents that are thermophilic.

### CROSS-REFERENCE TO RELATED APPLICATION

This application claims the benefit of U.S. Provisional Patent Application No. 63/411,965, filed Sep. 30, 2022, and entitled “Mesophilic Compositions for Nucleic Acid Amplification,” the disclosure of which is hereby incorporated by reference in its entirety

### FIELD

This disclosure relates to novel amplification compositions and methods, in particular for use in nucleic acid amplification and sequencing, preferably that do not involve reagents that are thermophilic.

### SEQUENCE LISTING

The instant application contains a Sequence Listing which has been submitted electronically in xml format and is hereby incorporated by reference in its entirety. Said xml copy was created on Sep. 22, 2023, is named 85491_08500_US.xml, and is 6.4 kilobytes in size.

### BACKGROUND

The detection of analytes such as nucleic acid sequences that are present in a biological sample has been used as a method for identifying and classifying microorganisms, diagnosing infectious diseases, detecting and characterising genetic abnormalities, identifying genetic changes associated with cancer, studying genetic susceptibility to disease, measuring response to various types of treatment and whole exome sequencing to name a few. A common technique for detecting nucleic acid sequences in a biological sample is nucleic acid amplification and sequencing.

Methods of nucleic acid amplification which allow amplification products to be immobilised on a solid support in order to form arrays comprised of clusters or “colonies” formed from a plurality of identical immobilised polynucleotide strands and a plurality of identical immobilised complementary strands are known. The nucleic acid molecules present in DNA colonies on the clustered arrays prepared according to these methods can provide templates for sequencing reactions.

One method for sequencing a polynucleotide template involves performing multiple extension reactions using a DNA polymerase to successively incorporate labelled nucleotides to a template strand. In such a “sequencing by synthesis” reaction a new nucleotide strand base-paired to the template strand is built up in the 5′ to 3′ direction by successive incorporation of individual nucleotides complementary to the template strand.

There remains a need to develop new amplification compositions and methods that increase throughput and accuracy of sequencing runs. The present disclosure addresses this need.

### SUMMARY

In one aspect of the disclosure, there is provided an amplification composition (also referred to herein as the composition) comprising ATP sulfurylase.

The amplification composition may comprise ATP sulfurylase at a concentration of about 0.01 pM to about 1000 μM, about 0.1 μM to about 500 μM, about 10 μM to about 200 μM, about 100 μM.

The amplification composition may further comprise adenylyl sulfate (APS). Preferably, the amplification composition comprises APS at a concentration of about 0.01 μM to about 1000 μM, about 0.1 μM to about 100 μM, about 0.5 μM to about 50 μM, about 1 μM to about 20 μM, or about 2 μM to about 10 μM.

The composition may also further comprise at least one selected from the group comprising a polymerase, a recombinase, a plurality of nucleotide triphosphates (NTPs) and a single stranded nucleotide binding (SSB) protein.

The composition may also further comprise at least one selected from the group comprising a recombinase, NTPs and a single stranded nucleotide binding (SSB) protein.

Preferably, the ATP sulfurylase is derived from a mesophilic organism, preferably Saccharomyces cerevisiae or E. coli. In one embodiment, the ATP sulfurylase comprises an amino acid sequence as defined in SEQ ID NO: 1 or a functional variant or fragment thereof.

Preferably, the polymerase is DNA Polymerase I and the recombinase is Recombinase A.

Preferably, the composition does not comprise PEG.

The amplification composition may also comprise a buffer, wherein preferably, the composition is buffered to a pH of about 6.0 to about 9.0, preferably about 6.5 to about 8.8, more preferably about 7.5 to about 8.7, even more preferably about 8.3 to about 8.6.

The amplification composition may be a clustering composition or a sequencing-by-synthesis composition or a resynthesis composition.

In another aspect, there is provided a kit comprising the amplification composition as defined herein.

The kits may further comprise a metal cofactor composition, preferably wherein the metal cofactor composition comprises magnesium ions.

In another aspect, there is provided the amplification composition or the kit to amplify a nucleic acid sequence and/or sequence a nucleic acid sequence.

In another aspect, there is provided the use of the amplification composition or the kit to form a monoclonal cluster of a template nucleic acid sequence.

In another aspect, there is provided a method of amplifying a target nucleic acid template, the method comprising adding the amplification composition to a sample comprising the nucleic acid template.

In another aspect, there is provided a method of forming a monoclonal cluster of a template nucleic acid sequence, the method comprising adding the amplification composition to a sample comprising the nucleic acid template.

Preferably, the nucleic acid amplification is performed at a temperature of between 20 and 45° C., preferably between 30 and 40° C.

Preferably, the method comprises adding the amplification composition only once.

In another aspect, there is provided a method of sequencing a nucleic acid sequence, wherein the method comprises:

amplifying a nucleic acid template using a method as recited above; and sequencing the amplified nucleic acid template.

Preferably the step of sequencing the amplified nucleic acid template comprises conducting a first sequencing read and a second sequencing read.

Preferably the step of sequencing the amplified nucleic acid template is conducted using a sequencing-by-synthesis technique or a sequencing-by-ligation technique.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIG. 1A shows a typical amplification mixture, such as exclusion amplification (ExAmp). Amplification mixtures typically use four key enzymes to cluster library specific DNA on a solid support, such as flow-cell; a recombinase, a DNA polymerase, a single-stranded DNA binding protein (SSB) and a creatine kinase. FIG. 1B shows the primer extension step. The primer extension step within the RPA (recombinase-polymerase amplification) reaction generates PPi from the DNA polymerase.

FIG. 2A again shows that the primer extension step within the RPA reaction generates PPi from the DNA polymerase. FIG. 2B shows that PPi can be enzymatically converted to ATP in the presence of the small molecule APS and ATP sulfurylase. FIG. 2C shows that the ATP is then used by the recombinase to form filaments for homology searching.

FIGS. 3A and 3B provide a strand invasion assay (SIA) overview. An annealed duplex where one strand (red) has a black hole quencher (BHQ) attached and a strand (black) where a fluorescent dye (FAM) is attached. An oligonucleotide (oligo; green) with a region of homology to the annealed duplex (red) is present in the reaction tube. Recombinase, and combinations of the (+/−) of the energy regeneration system can be supplemented to the reaction tube. A fluorescent signal is generated when the BHQ strand is removed by the invading unlabeled strand allowing the FAM dye to be excited.

FIG. 4 shows that HCXE supported RPA reactions in the presence and absence of Sce ATP sulfurylase. M; DNA ladder with the sizes noted; lane 1 is the positive control with all components of HCXE; lane 2 HCXE with the energy system CK/CP removed, but 4.4 mM ATP present in the system; lane 3 HCXE with the energy system CK/CP removed, 4.4 mM ATP, 2 μg Sce ATP Sulfurylase and 4 mM APS; lane 4 negative control, no HCXE; just PhiX template; lane 5 HCXE with the energy system CK/CP removed, 4.4 mM ATP, and 4 mM APS.

FIG. 5A.) Sequence Analysis Viewer (SAV) was utilized to extract the Read 1 (R1) and Read 2 (R2) intensities from the NextSeq 2000 runs. The striped black and white bar is R1 or R2 intensity of the control clustering formulation with a standard commercial recipe. The grey bar is R1 or R2 intensity using the clustering formulation supplemented with 0.3 U PPiase (pyrophosphatase) per 100μ1 clustering formulation with the standard modified recipe to pull from the unique well with the cartridge. The black bar is R1 or R2 intensity using a clustering formulation supplemented with 1.2 U of PPiase per 100 μl clustering formulation with the standard modified recipe to pull from the unique well with the cartridge. For both read 1 and read 2 the presence of the PPiase increased the intensity. Additionally, the increase in intensity was observed in a concentration dependent manner meaning that in increasing the amount of enzyme utilized increases the intensity signal for both read 1 and read 2. The unit of measure of intensity is relative fluorescence units (RFU). FIG. 5B.) The Quality Score represented in the % Q30 values extracted from SAV. The control bar is the standard clustering formulation; the grey bar is the clustering formulation supplemented with 0.3 U PPiase per 100 μl clustering formulation; the black bar is the clustering formulation 1.2 U PPiase per 100 μl clustering formulation. The % Q30> scores increased in the presence of the PPiase in a concentration dependent manner relative to the control. FIG. 5C.) Instrument yield measured in G output was extracted from SAV. The control bar is the standard clustering formulation; the grey bar is the clustering formulation supplemented with 0.3 U PPiase per 100 μl clustering formulation; the black bar is the clustering formulation 1.2 U PPiase per 100 μl clustering formulation. The yield of NextSeq 2000 increased in the presence of the PPiase in a concentration dependent manner relative to the control. FIG. 5D.) Percent passing filter clusters (% PF) was extracted from SAV. The control bar is the standard clustering formulation; the grey bar is the clustering formulation supplemented with 0.3 U PPiase per 100 μl clustering formulation; the black bar is the clustering formulation 1.2 U PPiase per 100 μl clustering formulation. The % PF of NextSeq 2000 increased in the presence of the PPiase in a concentration dependent manner relative to the control. FIG. 5E.) shows a reaction scheme for the enzymatic hydrolysis of inorganic pyrophosphate into orthophosphate by inorganic pyrophosphatase

### DETAILED DESCRIPTION

The following described features apply to all aspects and embodiments of the disclosure. The present disclosure is directed to amplification methods and compositions.

The present disclosure can be used in sequencing, for example pairwise sequencing. Methodology applicable to the present disclosure have been described in WO 08/041002, WO 07/052006, WO 98/44151, WO 00/18957, WO 02/06456, WO 07/107710, WO05/068656, U.S. Ser. No. 13/661,524 and US 2012/0316086, the contents of which are herein incorporated by reference. Further information can be found in US 20060024681, US 200602926U, WO 06110855, WO 06135342, WO 03074734, WO07010252, WO 07091077, WO 00179553 and WO 98/44152, the contents of which are herein incorporated by reference.

Sequencing generally comprises four fundamental steps: 1) library preparation to form a plurality of template molecules available for sequencing; 2) cluster generation to form an array of amplified single template molecules on a solid support; 3) sequencing the cluster array; and 4) data analysis to determine the target sequence.

Library preparation is the first step in any high-throughput sequencing platform. During library preparation, nucleic acid sequences, for example genomic DNA sample, or cDNA or RNA sample, is converted into a sequencing library, which can then be sequenced. By way of example with a DNA sample, the first step in library preparation is random fragmentation of the DNA sample. Sample DNA is first fragmented and the fragments of a specific size (typically 200-500 bp, but can be larger) are ligated, sub-cloned or “inserted” in-between two oligo adapters (adapter sequences). This may be followed by amplification and sequencing. The original sample DNA fragments are referred to as “inserts”. Alternatively “tagmentation” can be used to attach the sample DNA to the adapters. In tagmentation, double-stranded DNA is simultaneously fragmented and tagged with adapter sequences and PCR primer binding sites. The combined reaction eliminates the need for a separate mechanical shearing step during library preparation. The target polynucleotides may advantageously also be size-fractionated prior to modification with the adaptor sequences.

As used herein an “adapter” sequence comprises a short sequence-specific oligonucleotide that is ligated to the 5′ and 3′ ends of each DNA (or RNA) fragment in a sequencing library as part of library preparation. The adaptor sequence may further comprise non-peptide linkers.

As will be understood by the skilled person, a double-stranded nucleic acid will typically be formed from two complementary polynucleotide strands comprised of deoxyribonucleotides joined by phosphodiester bonds, but may additionally include one or more ribonucleotides and/or non-nucleotide chemical moieties and/or non-naturally occurring nucleotides and/or non-naturally occurring backbone linkages. In particular, the double-stranded nucleic acid may include non-nucleotide chemical moieties, e.g. linkers or spacers, at the 5′ end of one or both strands. By way of non-limiting example, the double-stranded nucleic acid may include methylated nucleotides, uracil bases, phosphorothioate groups, also peptide conjugates etc. Such non-DNA or non-natural modifications may be included in order to confer some desirable property to the nucleic acid, for example to enable covalent, non-covalent or metal-coordination attachment to a solid support, or to act as spacers to position the site of cleavage an optimal distance from the solid support. A single stranded nucleic acid consists of one such polynucleotide strand. Where a polynucleotide strand is only partially hybridised to a complementary strand—for example, a long polynucleotide strand hybridised to a short nucleotide primer—it may still be referred to herein as a single stranded nucleic acid.

In one embodiment, the template comprises, in the 5′ to 3′ direction, a first primer-binding sequence (e.g. P5, e.g. SEQ ID NO: 2), an index sequence (e.g. i5), a first sequencing binding site (e.g. SB S3), an insert, a second sequencing binding site (e.g. SBS12), a second index sequence (e.g. i7) and a second primer-binding sequence (e.g. P7′ .g. SEQ ID NO: 5). In another embodiment, the template comprises, in the 3′ to 5′ direction, a first primer-binding site (e.g. P5′ .g. SEQ ID NO: 4, which is complementary to P5), an index sequence (e.g. i5′, which is complementary to IS), a first sequencing binding site (e.g. SBS3′ which is complementary to SBS3), an insert, a second sequencing binding site (e.g. SBS12′, which is complementary to SBS12), a second index sequence (e.g. i7′, which is complementary to 17) and a second primer-binding sequence (e.g. P7, .g. SEQ ID NO: 3 which is complementary to P7′). Either template is referred to herein as a “template strand” or “a single stranded template”. Both template strands annealed together is referred to herein as “a double stranded template”.

A sequence comprising at least a primer-binding sequence (preferably a combination of a primer-binding sequence, an index sequence and a sequencing binding site) is referred to herein as an adaptor sequence, and a single insert is flanked by a 5′ adaptor sequence and a 3′ adaptor sequence. The first primer-binding sequence may also comprise a sequencing primer for the index read (IS). “Primer-binding sequences” may also be referred to as “clustering sequences” “clustering primers” “primers” or “cluster primers” in the present disclosure, and such terms may be used interchangeably.

In a further embodiment, the P5′ and P7′ primer-binding sequences are complementary to short primer sequences (or lawn primers) present on the surface of the flow cells. Binding of P5′ and P7′ to their complements (P5 and P7) on—for example—the surface of the flow cell, permits nucleic acid amplification. As used herein “′” denotes the complementary strand.

The primer-binding sequences in the adaptor which permit hybridisation to amplification primers (e.g. lawn primers) will typically be around 20-40 nucleotides in length, although, in embodiments, the disclosure is not limited to sequences of this length. The precise identity of the amplification primers (e.g. lawn primers), and hence the cognate sequences in the adaptors, are generally not material to the disclosure, as long as the primer-binding sequences are able to interact with the amplification primers in order to direct PCR amplification. The sequence of the amplification primers may be specific for a particular target nucleic acid that it is desired to amplify, but in other embodiments these sequences may be “universal” primer sequences which enable amplification of any target nucleic acid of known or unknown sequence which has been modified to enable amplification with the universal primers. The criteria for design of PCR primers are generally well known to those of ordinary skill in the art.

The index sequences (also known as a barcode or tag sequence) are unique short DNA (or RNA) sequences that are added to each DNA (or RNA) fragment during library preparation. The unique sequences allow many libraries to be pooled together and sequenced simultaneously. Sequencing reads from pooled libraries are identified and sorted computationally, based on their barcodes, before final data analysis. Library multiplexing is also a useful technique when working with small genomes or targeting genomic regions of interest. Multiplexing with barcodes can exponentially increase the number of samples analysed in a single run, without drastically increasing run cost or run time. Examples of tag sequences are found in WO05068656, whose contents are incorporated herein by reference in their entirety. The tag can be read at the end of the first read, or equally at the end of the second read, for example using a sequencing primer complementary to the strand marked P7. The disclosure is not limited by the number of reads per cluster, for example two reads per cluster: three or more reads per cluster are obtainable simply by dehybridising a first extended sequencing primer, and rehybridising a second primer before or after a cluster repopulation/strand resynthesis step. Methods of preparing suitable samples for indexing are described in, for example US60/899221. Single or dual indexing may also be used. With single indexing, up to 48 unique 6-base indexes can be used to generate up to 48 uniquely tagged libraries. With dual indexing, up to 24 unique 8-base Index 1 sequences and up to 16 unique 8-base Index 2 sequences can be used in combination to generate up to 384 uniquely tagged libraries. Pairs of indexes can also be used such that every i5 index and every i7 index are used only one time. With these unique dual indexes, it is possible to identify and filter indexed hopped reads, providing even higher confidence in multiplexed samples.

The sequencing binding sites are sequencing and/or index primer binding sites and indicates the starting point of the sequencing read. During the sequencing process, a sequencing primer anneals (i.e. hybridises) to a portion of the sequencing binding site on the template strand. The polymerase enzyme binds to this site and incorporates complementary nucleotides base by base into the growing opposite strand. In one embodiment, the sequencing process comprises a first and second sequencing read. The first sequencing read may comprise the binding of a first sequencing primer (read 1 sequencing primer) to the first sequencing binding site (e.g. SBS3′) followed by synthesis and sequencing of the complementary strand. This leads to the sequencing of the insert. In a second step, an index sequencing primer (e.g. i7 sequencing primer) binds to a second sequencing binding site (e.g. SBS12) leading to synthesis and sequencing of the index sequence (e.g. sequencing of the i7 primer). The second sequencing read may comprise binding of an index sequencing primer (e.g. i5 sequencing primer) to the complement of the first sequencing binding site on the template (e.g. SBS3) and synthesis and sequencing of the index sequence (e.g. i5). In a second step, a second sequencing primer (read 2 sequencing primer) binds to the complement of the primer (e.g. i7 sequencing primer) binds to a second sequencing binding site (e.g. SBS12′) leading to synthesis and sequencing of the insert in the reverse direction.

Once a double stranded nucleic acid template library is formed, typically, the library has previously been subjected to denaturing conditions to provide single stranded nucleic acids. Suitable denaturing conditions will be apparent to the skilled reader with reference to standard molecular biology protocols (Sambrook et al., 2001, Molecular Cloning, A Laboratory Manual, 3rd Ed, Cold Spring Harbor Laboratory Press, Cold Spring Harbor Laboratory Press, NY; Current Protocols, eds Ausubel et al). In one embodiment, chemical denaturation is used.

Following denaturation, a single-stranded template library can be contacted in free solution onto a solid support comprising surface capture moieties (for example P5 and P7 LAWN primers). This solid support is typically a flowcell, although in alternative embodiments, seeding and clustering can be conducted off-flowcell using other types of solid support.

By way of brief example, following attachment of the P5 and P7 primers, the solid support may be contacted with the template to be amplified under conditions which permit hybridisation (or annealing—such terms may be used interchangeably) between the template and the immobilised primers. The template is usually added in free solution under suitable hybridisation conditions, which will be apparent to the skilled reader. Typically, hybridisation conditions are, for example, 5×SSC at 40° C. However, other temperatures may be used during hybridisation, for example about 50° C. to about 75° C., about 55° C. to about 70° C., or about 60° C. to about 65° C. Solid-phase amplification can then proceed. The first step of the amplification is a primer extension step in which nucleotides are added to the 3′ end of the immobilised primer using the template to produce a fully extended complementary strand. The template is then typically washed off the solid support. The complementary strand will include at its 3′ end a primer-binding sequence (i.e. either P5′ or P7′) which is capable of bridging to the second primer molecule immobilised on the solid support and binding. Further rounds of amplification (analogous to a standard PCR reaction) lead to the formation of (monoclonal) clusters or colonies of template molecules bound to the solid support. This is called clustering.

Thus, solid-phase amplification by either the method analogous to that of WO 98/44151 or that of WO 00/18957 (the contents of which are incorporated herein in their entirety by reference) will result in production of a clustered array comprised of colonies of “bridged” amplification products. Both strands of the amplification products will be immobilised on the solid support at or near the 5′ end, this attachment being derived from the original attachment of the amplification primers. Typically, the amplification products within each colony will be derived from amplification of a single template (target) molecule. Other amplification procedures may be used, and will be known to the skilled person. For example, amplification may be isothermal amplification using a strand displacement polymerase; or may be exclusion amplification as described in WO 2013/188582. Further information on amplification can be found in WO0206456 and WO07107710, the contents of which are incorporated herein in their entirety by reference. Through such approaches, a cluster of single template molecules is formed.

To facilitate sequencing, it is preferable if one of the strands is removed from the surface to allow efficient hybridisation of a sequencing primer to the remaining immobilised strand. Suitable methods for linearisation are described in more detail in application number WO07010251, the contents of which are incorporated herein by reference in their entirety.

Sequence data can be obtained from both ends of a template duplex by obtaining a sequence read from one strand of the template from a primer in solution, copying the strand using immobilised primers, releasing the first strand and sequencing the second, copied strand. For example, sequence data can be obtained from both ends of the immobilised duplex by a method wherein the duplex is treated to free a 3′-hydroxyl moiety that can be used an extension primer. The extension primer can then be used to read the first sequence from one strand of the template. After the first read, the strand can be extended to fully copy all the bases up to the end of the first strand. This second copy remains attached to the surface at the 5′-end. If the first strand is removed from the surface, the sequence of the second strand can be read. This gives a sequence read from both ends of the original fragment.

Sequencing can be carried out using any suitable “sequencing-by-synthesis” technique, wherein nucleotides are added successively to the free 3′ hydroxyl group, resulting in synthesis of a polynucleotide chain in the 5′ to 3′ direction. The nature of the nucleotide added is preferably determined after each addition. One particular sequencing method relies on the use of modified nucleotides that can act as reversible chain terminators. Such reversible chain terminators comprise removable 3′ blocking groups. Once such a modified nucleotide has been incorporated into the growing polynucleotide chain complementary to the region of the template being sequenced there is no free 3′-OH group available to direct further sequence extension and therefore the polymerase cannot add further nucleotides. Once the nature of the base incorporated into the growing chain has been determined, the 3′ block may be removed to allow addition of the next successive nucleotide. By ordering the products derived using these modified nucleotides it is possible to deduce the DNA sequence of the DNA template. Such reactions can be done in a single experiment if each of the modified nucleotides has attached thereto a different label, known to correspond to the particular base, to facilitate discrimination between the bases added at each incorporation step. Suitable labels are described in PCT application PCT/GB/2007/001770, the contents of which are incorporated herein by reference in their entirety. Alternatively, a separate reaction may be carried out containing each of the modified nucleotides added individually.

The modified nucleotides may carry a label to facilitate their detection. In a particular embodiment, the label is a fluorescent label. Each nucleotide type may carry a different fluorescent label. However the detectable label need not be a fluorescent label. Any label can be used which allows the detection of the incorporation of the nucleotide into the DNA sequence. One method for detecting the fluorescently labelled nucleotides comprises using laser light of a wavelength specific for the labelled nucleotides, or the use of other suitable sources of illumination. The fluorescence from the label on an incorporated nucleotide may be detected by a CCD camera or other suitable detection means. Suitable detection means are described in PCT/US2007/007991, the contents of which are incorporated herein by reference in their entirety.

Alternative methods of sequencing include sequencing by ligation, for example as described in U.S. Pat. No. 6,306,597 or WO06084132, the contents of which are incorporated herein by reference.

In some embodiments, sequencing may involve pairwise sequencing. The typical steps of pairwise sequencing are known and have been described in WO 2008/041002, the contents of which are herein incorporated by reference. However, the key steps will be briefly described.

The disclosure relates to methods for sequencing two regions of a target double-stranded polynucleotide template, referred to herein as the first and second regions for sequence determination. The first and second regions for sequence determination are at both ends of complementary strands of the double-stranded polynucleotide template, which are referred to herein respectively as first and second template strands. Once the sequence of a strand is known, the sequence of its complementary strand is also known, therefore the term two regions can apply equally to both ends of a single stranded template, or both ends of a double stranded template, wherein a first region and its complement are known, and a second region and its complement are known.

A plurality of template polynucleotide duplexes are immobilised on a solid support. The template polynucleotides may be immobilised in the form of an array of amplified single template molecules, or ‘clusters’. Each of the duplexes within a particular cluster comprises the same double-stranded target region to be sequenced. The duplexes are each formed from complementary first and second template strands which are linked to the solid support at or near to their 5′ ends. Typically, the template polynucleotide duplexes will be provided in the form of a clustered array.

An alternate starting point is a plurality of single stranded templates which are attached to the same surface as a plurality of primers that are complementary to the 3′ end of the immobilised template. The primers may be reversibly blocked to prevent extension. The single stranded templates may be sequenced using a hybridised primer at the 3′ end. The sequencing primer may be removed after sequencing, and the immobilised primers deblocked to release an extendable 3′ hydroxyl. These primers may be used to copy the template using bridged strand resynthesis to produce a second immobilised template that is complementary to the first. Removal of the first template from the surface allows the newly single stranded second template to be sequenced, again from the 3′ end. Thus, both ends of the original immobilised template can be sequenced. Such a technique allows paired end reads where the templates are amplified using a single extendable immobilised primer, for example as described in Polony technology (Nucleic Acids Research 27, 24, e34(1999)) or emulsion PCR (Science 309, 5741, 1728-1732 (2005); Nature 437, 376-380 (2005)).

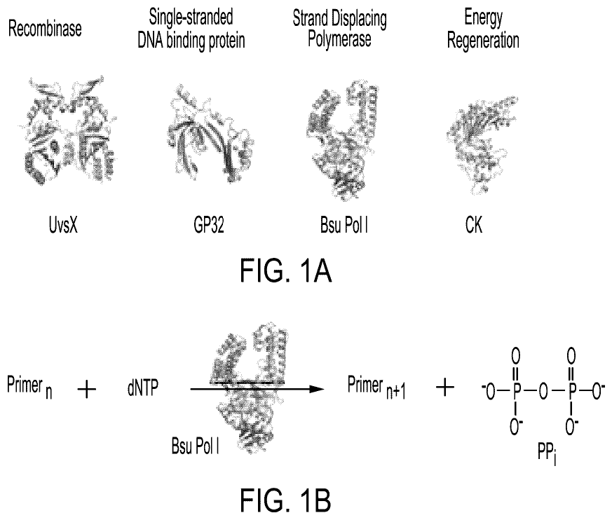

A critical step in nucleic acid sequencing is amplification, and in particular in the generation of the clusters that comprise an array (or clonal cluster) of amplified template molecules on a solid support. The amplification or clustering reaction typically uses four enzymes, which facilitate clustering, for example through an isothermal system, such as recombinase-polymerase amplification or RPA (FIG. 1a).

A key step in template amplification is primer extension. Typically this is performed by a polymerase, such as Bacillus subtilus (Bsu) DNA polymerase I (Pol), which generates a by-product called inorganic pyrophosphate (PPi) with each successive NTP (e.g. dNTP) incorporation event (as shown in FIG. 1b). The liberation of PPi is essential to primer extension, however, when it builds up in the aqueous environment of the in vitro reaction it can inhibit the reaction. Accumulation of PPi can also stall the DNA polymerase during strand synthesis, thereby limiting the forward reaction. This is especially problematic when the polymerase encounters secondary structural features within the amplifying DNA strand, such as a G-quadruplex. Stalling of the DNA polymerase can also lead to a phenotype where parts of the library are not clustered and therefore not ultimately sequenced. Finally, the accumulation of PPi can affect the ability of accurately call/detect insertion/deletion events (INDELS) and variants in secondary metrics.

As used herein, the term “inorganic pyrophosphate” (or “PPi”) may refer to two phosphate residues connected by a phosphoanhydride bond.

An inorganic pyrophosphate may be present in an acid form, a salt form, or a combination thereof. In cases where the inorganic pyrophosphate is present in a salt form, the inorganic pyrophosphate may comprise a cation (not including H⁺). For example, the cation may be selected from “metal cations” or “non-metal cations”. Metal cations may include alkali metal ions (e.g. lithium, sodium, potassium, rubidium or caesium ions). Non-metal cations may include ammonium salts (e.g. alkylammonium salts) or phosphonium salts (e.g. alkylphosphonium salts).

The inorganic pyrophosphate may be soluble in aqueous medium.

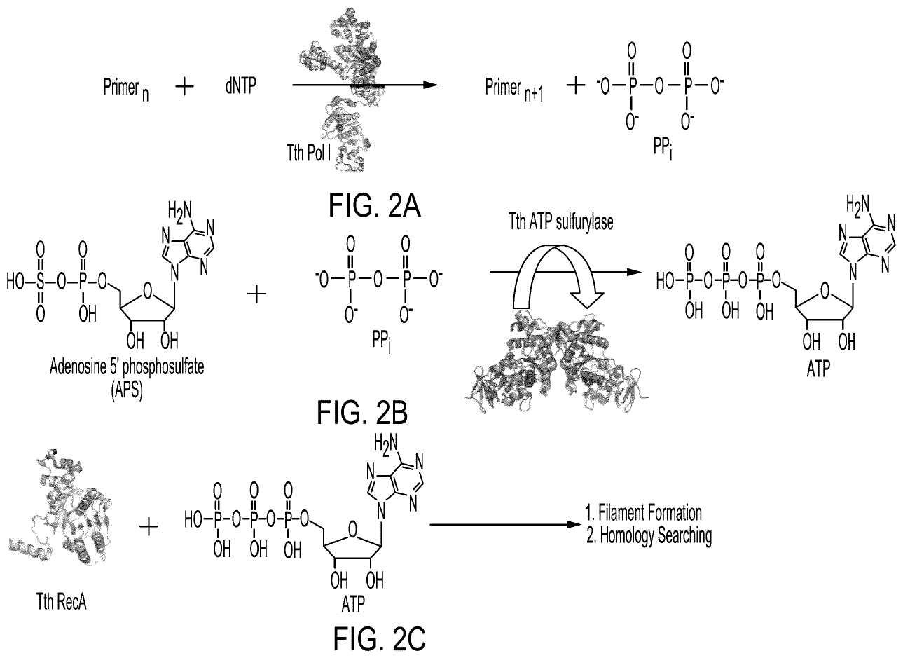

The present disclosure provides a method to remove or reduce the amount of PPi in the clustering reaction. One way to remove PPi generated from the primer extension reaction shown in FIG. 2a, is to convert the PPi molecule enzymatically into ATP by a transferase reaction mediated by ATP sulfurylase, as shown in FIG. 2b. Not only does this reaction consume the inhibitory waste product, PPi, but as an additional benefit, the ATP generated can be used as an energy source for the recombinase, as shown in FIG. 2c. This in turn has been found to improve clustering kinetics and allow the amplification (and subsequent sequencing) of difficult regions of the genome.

Where the amplification composition comprises creatine kinase (CK)/creatine phosphate (CP), ATP sulfurylase/APS can be additionally used. Alternatively, ATP sulfurylase/APS can be used instead of creatine kinase (CK)/creatine phosphate (CP).

Accordingly, in one aspect, there is provided an amplification composition comprising means to reduce or remove inhibitory PPi from the system. By “reduce” is meant that the amount or concentration of PPi at any given time point is reduced in a system comprising the amplification composition by at least 10%, 20%, 30%, 40%, 50%, 60%, 70%, 80%, 90%, 95% or 99% compared to a system at the same time point that does not comprise the amplification composition. By “remove” is meant that any PPi generated by the polymerase is removed/converted by the amplification composition such that PPi is not or is barely detectable at any given time point in the system.

In one embodiment, the amplification composition is a clustering composition.

As used herein, the term “cluster” may refer to a clonal group of template polynucleotides (e.g. DNA or RNA) bound within a single well of a flowcell. A “cluster” may contain a sufficient number of copies of a single template polynucleotide such that the cluster is able to output a signal (e.g. a light signal) that allows a single sequencing read to be performed on the cluster. A “cluster” may comprise, for example, about 500 to about 2000 copies, preferably about 600 to about 1800 copies, more preferably about 700 to about 1600 copies, even more preferably about 800 to 1400 copies, yet even more preferably about 900 to 1200 copies, most preferably about 1000 copies of a single template polynucleotide. The copies of the single template polynucleotide may comprise at least about 50%, preferably at least about 60%, more preferably at least about 70%, even more preferably at least about 80%, yet even more preferably at least about 90%, most preferably about 95%, 98%, 99% or 100% of all polynucleotides within a single well of the flowcell, and thus providing a substantially monoclonal “cluster”.

The development of new clustering compositions can be more challenging, as other factors need to be taken into consideration (e.g. maintaining monoclonality and a high density/intensity of individual clusters).

Accordingly, in one aspect, there is provided an amplification (or clustering) composition comprising an ATP sulfurylase.

As used herein, an “ATP sulfurylase” catalyzes the conversion of PPi and adenylyl sulfate (APS) into ATP (and sulfate), as shown below:

APS+PPi→ATP

In one embodiment, the amplification composition comprises an ATP sulfurylase at a concentration of about 0.01 μM to about 1000 μM, about 0.1 μM to about 100 μM, about 0.5 μM to about 50 μM, about 1 μM to about 20 μM, or about 2 μM to about 10 μM. Alternatively, the amplification composition comprises between about 0.01 U/μL and about 100 U/μL of the ATP sulfurylase, between about 0.1 U/μL and about 50 U/μL, between about 0.2 U/μL and about 30 U/μL, between about 0.3 U/μL and about 20 U/μL, between about 0.5 U/μL and about 10 U/μL, or between about 1.0 U/μL and about 5.0 U/μL. For example, the amplification composition may comprise around 0.3 U/μL, 0.4 U/μL, 0.5 U/μL, 0.6 U/μL, 0.7 U/μL, 0.8 U/μL, 0.9 U/μL, 1.0 U/μL, 1.1 U/μL, 1.2 U/μL, 1.3 U/μL, 1.4 U/μL, 1.5 U/μL, 1.6 U/μL, 1.7 U/μL, 1.8 U/μL, 1.9 U/μL or around 2.0 U/μL of the ATP sulfurylase. Alternatively, the ATP sulfurylase is present at a wt % between about 0.01 wt % to about 5.0 wt %, about 0.02 wt % to about 4.5 wt %, about 0.05 wt % to about 4.0 wt %, about 0.08 wt % to about 3.5 wt %, about 0.1 wt % to about 3.0 wt %, about 0.2 wt % to about 2.5 wt %, or about 0.5 wt % to about 2.0 wt % with respect to a total wt % of the amplification composition by dry mass.

As described herein, the ATP sulfurylase is derived from a mesophile.

As used herein, the term “mesophile” may refer to a protein that does not substantially denature at moderate temperatures, for example, between 20 and 45° C. These proteins likely have an optimum activity in the range 30 to 40° C.

Accordingly, in an alternative embodiment, the ATP sulfurylase may have an optimum working temperature of about 30° C. to 40° C. Preferably, the ATP sulfurylase may have an optimum working temperature of about 30° C. to about 40° C., preferably about 32° C. to about 39° C., more preferably about 34° C. to about 38° C.; for example, the ATP sulfurylase may have an optimum working temperature of about 30° C., about 35° C., about 37° C., or about 40° C.

As used herein, the term “optimum working temperature” may refer to a temperature at which the catalytic activity of the enzyme reaches a peak maximum value.

As such, the disclosure provides a mesophilic amplification composition, wherein the amplification composition comprises a mesophilic ATP sulfurylase.

In a preferred embodiment, the ATP sulfurylase is derived from a mesophile. Examples of a mesophile include Saccharomyces cerevisiae and E. coli. In one embodiment, the inorganic ATP sulfurylase comprises the sequence as shown in SEQ ID NO: 1 or a functional variant or functional fragment thereof:

(SEQ ID NO: 1)
MNTALAQQIANEGGVEAWMIAQQHKSLLRFLTCGSVDDGKS
TLIGRLLHDTRQIYEDQLSSLHNDSKRHGTQGEKLDLALL
VDGLQAEREQGITIDVAYRYFSTEKRKFIIADTPGHEQYT
RNMATGASTCELAILLIDARKGVLDQTRRHSFISTLLGIK
HLVVAINKMDLVDYSEETFTRIRKDYLTFAEQLPGNLDIR
FVPLSALEGDNVASQSESMPWYSGPTLLEVLETVEIQRVV
DAQPMRFPVQYVNRPNLDFRGYAGTLASGRVEVGQRVKVL
PSGVESNVARIVTFDGDREEAFAGEAITLVLTDEIDISRG
DLLLAADEALPAVQSASVDVVWMAEQPLSPGQSYDIKIAG
KKTRARVDGIRYQVDINNLTQREVENLPLNGIGLVDLTFD
EPLVLDRYQQNPVTGGLIFIDRLSNVTVGAGMVHEPVSQA
TAAPSEFSAFELELNALVRRHFPHWGARDLLGDK

In a further embodiment, the amplification composition further comprises 5′-adenosine phosphosulfate (APS). Accordingly, the amplification composition comprises ATP sulfurylase and APS.

As used herein, the term “APS” (or “5′-adenosine phosphosulfate”, “adenylyl sulfate”) refers to a compound comprising the following structure:

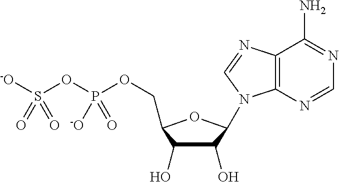

APS may be present in an acid form (where counterion may be H⁺), a salt form (where the counterion may be a cation not including H⁺), or a combination thereof (where the counterion may be a mixture of H⁺ and cations not including H⁺). For example, the cation may be selected from “metal cations” or “non-metal cations”. Metal cations may include alkali metal ions (e.g. lithium, sodium, potassium, rubidium or caesium ions). Non-metal cations may include ammonium salts (e.g. alkylammonium salts) or phosphonium salts (e.g. alkylphosphonium salts).

In one embodiment, the amplification composition comprises APS at a concentration of about 0.01 μM to about 1000 μM, about 0.1 μM to about 100 μM, about 0.5 μM to about 50 μM, about 1 μM to about 20 μM, or about 2 μM to about 10 μM. Alternatively, the APS is present at a wt % between about 0.01 wt % to about 5.0 wt %, about 0.02 wt % to about 4.5 wt %, about 0.05 wt % to about 4.0 wt %, about 0.08 wt % to about 3.5 wt %, about 0.1 wt % to about 3.0 wt %, about 0.2 wt % to about 2.5 wt %, or about 0.5 wt % to about 2.0 wt % with respect to a total wt % of the amplification composition by dry mass.

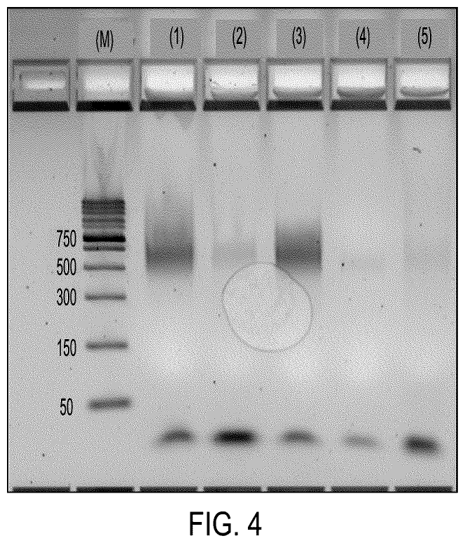

As shown in FIG. 4, the present inventor has found that the generation of ATP during amplification by the ATP sulfurylase, provides a source of ATP for use by the recombinase to perform homology searching and strand invasion.

The present inventor also found that the removal of inorganic pyrophosphate from the amplification composition, for example by the addition of ATP sulfurylase, has a number of advantages in methods of nucleic acid amplification and sequencing. Specifically, the present inventor has found that the removal of inorganic pyrophosphate can be used to improve clustering kinetics, and in turn reduce clustering times (and thus turnaround times) and/or increase the signal intensities (and thus increase the sequence signal:noise ratios).

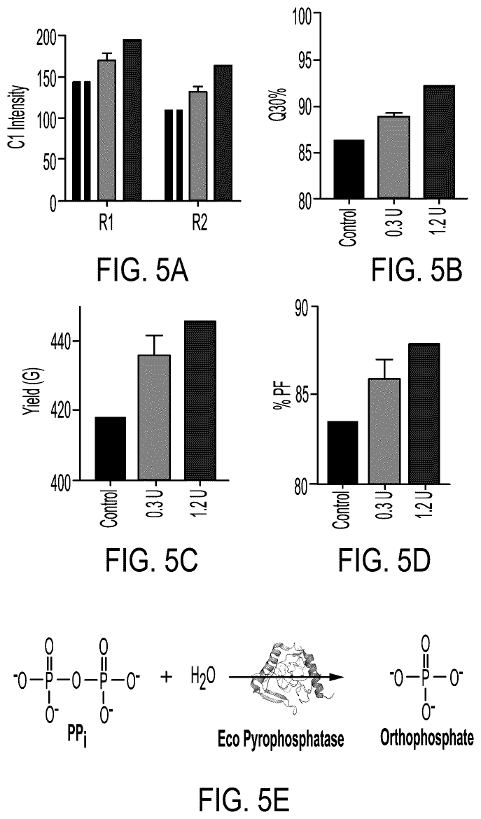

Specifically, the present inventor has found that improving clustering kinetics by the removal or reduction of PPi leads to improvements in sequencing performance, including, but not limited to, an increase in intensity, % PF, Q30 and Yield (g). By “% PF” is meant the % of reads that pass the chastity filter (chastity is the ratio is the ratio of the brightest base intensity divided by the sum of the brightest and second brightest base intensities“). By “Q30” is meant The percentage of bases with a quality score of 30 or higher. A quality score is “A quality score is an estimate of the probability of that base being called wrongly: q=−10×log10(p)”. By “yield” is meant the number of bases generated in the run. This is shown in FIG. 5. Here the addition of inorganic pyrophosphatase, as a reference means to reduce to PPi (as shown in FIG. 5E), at 0.3U and 1.2U increasing the intensity (FIG. 5A), % PF (FIG. 5B), Q30 (FIG. 5C) and Yield (g)(FIG. 5D).

In addition, increasing clustering intensity also allows amplification/clustering to take place in smaller wells, where a decrease in well size requires an increase in signal intensity.

Furthermore, as explained above, the accumulation of inorganic pyrophosphate stalls DNA polymerase. This is problematic where the DNA polymerase encounters structured secondary features like a G-quadruplex, leading to parts of the library that are not clustered/amplified and therefore not sequenced. Removal of inorganic pyrophosphate prevents stalling of the DNA polymerase, and consequently a decrease in sequence specific errors because the polymerase is able to cluster/amplify structured regions of the genome.

Finally, in addition to improving clustering kinetics (e.g. clustering times and the signal intensity), the addition of ATP sulfurylase can also significantly reduce the amount of clustering/amplification reagents needed by as much as 50%. As mentioned, in an amplification or clustering reaction it may be necessary to add the amplification composition more than once (the number of times the amplification composition is added to the flowcell may be called a “push”). Multiple pushes may be necessary to achieve the required level of sequence signal intensity. The removal of inorganic pyrophosphate can significantly increases the sequence signal intensity with a single push. Accordingly, by reducing PPi levels it is possible to additionally half the amount of amplification composition needed (i.e. half the COGs (cost of goods) without affecting clustering/amplification intensities.

By “amplification composition” is meant a composition that is suitable for the amplification of a target nucleic acid template. By contrast, a “cluster composition” refers to a composition that is suitable for the amplification of a (single) target sequence into a cluster (i.e. the composition is suitable for cluster generation, particularly for the generation of a monoclonal cluster) as described above, not just for any amplification method. In one embodiment, the composition is not additionally suitable for the detection or sequencing of the nucleic acid template. For example, in one embodiment, the composition does not comprise a fluorescent entity, such as probes, nucleotides labelled with a fluorescent entity, and/or primers labelled with a fluorescent entity. Alternatively, the composition does not comprise leuco dyes/reagents labelled with leuco dyes.

In one embodiment, the composition may be a re-synthesis composition. By re-synthesis is meant the step between the first and second sequencing reads where the template is copied using bridged strand resynthesis to produce a second immobilised template that is complementary to the first. Accordingly, the same composition as described herein may be used in resynthesis.

The composition may further comprise a recombinase.

As used herein, the term “recombinase” may refer to an enzyme which can facilitate invasion of a target nucleic acid by a polymerase and extension of a primer by the polymerase using the target nucleic acid as a template for amplicon formation. This process can be repeated as a chain reaction where amplicons produced from each round of invasion/extension serve as templates in a subsequent round. The process can occur more rapidly than standard PCR since a denaturation cycle (e.g. via heating or chemical denaturation) is not required. As such, recombinase-facilitated amplification can be carried out isothermally. It is generally desirable to include ATP, or other nucleotides (or in some cases non-hydrolysable analogs thereof) in a recombinase-facilitated amplification reagent to facilitate amplification. A mixture of recombinase and single-stranded binding (SSB) protein is particularly useful as SSB can further facilitate amplification. Recombinases may include, for example, RecA protein, the T4 uvsX protein, any homologous protein or protein complex from any phyla, or functional variants thereof. Eukaryotic RecA homologues are generally named Rad51 after the first member of this group to be identified. Other non-homologous recombinases may be utilised in place of RecA, for example, RecT or RecO.

The composition may further comprise a single-stranded nucleotide binding protein.

As used herein, the term “single-stranded nucleotide binding protein” may refer to any protein having a function of binding to a single stranded nucleic acid, for example, to prevent premature annealing, to protect the single-stranded nucleic acid from nuclease digestion, to remove secondary structure from the nucleic acid, or to facilitate replication of the nucleic acid. The term is intended to include, but is not necessarily limited to, proteins that are formally identified as Single Stranded Binding proteins by the Nomenclature Committee of the International Union of Biochemistry and Molecular Biology (NC-IUBMB). Exemplary single stranded binding proteins include, but are not limited to E. coli SSB, T4 gp32, T7 gene 2.5 SSB, phage phi 29 SSB, any homologous protein or protein complex from any phyla, or functional variants thereof.

The composition may further comprise a polymerase. Preferably, the polymerase may be a strand-displacing polymerase. In some preferred embodiments, the polymerase may be a DNA polymerase. In other preferred embodiments, the polymerase may be a RNA polymerase.

As used herein, the term “polymerase” may refer to an enzyme that produces a complementary replicate of a nucleic acid molecule using the nucleic acid as a template strand. Typically, DNA polymerases bind to the template strand and then move down the template strand sequentially adding nucleotides to the free hydroxyl group at the 3′ end of a growing strand of nucleic acid. DNA polymerases typically synthesise complementary DNA molecules from DNA templates and RNA polymerases typically synthesise RNA molecules from DNA templates (transcription). Polymerases can use a short RNA or DNA strand, called a primer, to begin strand growth. Some polymerases can displace the strand upstream of the site where they are adding bases to a chain. Such polymerases are said to be strand displacing, meaning they have an activity that removes a complementary strand from a template strand being read by the polymerase. Exemplary polymerases having strand displacing activity include, without limitation, the large fragment of Bst (Bacillus stearothermophilus) polymerase, exo-Klenow polymerase or sequencing grade T7 exo-polymerase. Some polymerases degrade the strand in front of them, effectively replacing it with the growing chain behind (5′ exonuclease activity). Some polymerases have an activity that degrades the strand behind them (3′ exonuclease activity). Some useful polymerases have been modified, either by mutation or otherwise, to reduce or eliminate 3′ and/or 5′ exonuclease activity.

The composition may further comprise a nucleotide triphosphate (NTP). Preferably, the nucleotide triphosphate may be a deoxynucleotide triphosphate (dNTP). More preferably, the composition comprises a plurality of NTPs or dNTPs, and preferably a mixture—for example comprising a plurality of dATP, dGTP, dCTP and dTTP for DNA clustering/synthesis or ATP, GTP, CTP and UTP for RNA clustering/synthesis. In one embodiment, the concentration of dNTPs may be between 0.1 and 2 mM, preferably between 0.2 to 1.5 mM, more preferably between 0.3 to 1.2 mM, even more preferably between 0.3 to 0.6 mM; for example, the concentration may be selected from 0.3 mM, 0.6 mM and 1.2 mM.

As used herein, the term “nucleotide triphosphate” may refer to a molecule containing a nitrogenous base (e.g. adenine, thymine, cytosine, guanine, uracil) bound to a 5-carbon sugar (e.g. ribose or deoxyribose), with three phosphate groups bound to the sugar.

As used herein, the term “deoxynucleotide triphosphate” or (dNTPs) may refer to a molecule containing a nitrogenous base (e.g. adenine, thymine, cytosine, guanine, uracil) bound to deoxyribose, with three phosphate groups bound to the deoxyribose.

The composition may further comprise APS.

The composition may further comprise another ATP-generating substrate (e.g. not including APS).

As used herein, the term “ATP-generating substrate” may refer to any substrate that is able to react with ADP to form ATP. Examples of ATP-generating substrates include creatine phosphate (CP).

The composition may further comprise another ATP-generating enzyme (e.g. not including ATP sulfurylase).

As used herein, the term “ATP-generating enzyme” may refer to any enzyme that is able to catalyse a reaction of ADP to form ATP. Examples of ATP-generating enzymes include creatine kinase.

The ATP-generating substrate as described herein may be paired with an appropriate ATP-generating enzyme that catalyses the reaction of that ATP-generating substrate with ADP to form ATP. Thus, in some preferred embodiments, the composition may comprise both APS and ATP sulfurylase, and/or both creatine phosphate (CP) and creatine kinase (CK).

In some embodiments, the composition may not comprise creatine kinase and/or creatine phosphate.

The composition may comprise ATP sulfurylase, and at least one selected from the group consisting of: a recombinase, a single-stranded nucleotide binding protein, a polymerase, nucleotide triphosphates (NTPs), and APS. Preferably, the composition may comprise ATP sulfurylase, and at least two selected from the group consisting of: a recombinase, a single-stranded nucleotide binding protein, a polymerase, nucleotide triphosphates (NTPs), and APS. More preferably, the composition may comprise ATP sulfurylase, and at least three selected from the group consisting of: a recombinase, a single-stranded nucleotide binding protein, a polymerase, nucleotide triphosphates (NTPs), and APS. Even more preferably, the composition may comprise ATP sulfurylase, and at least four selected from the group consisting of: a recombinase, a single-stranded nucleotide binding protein, a polymerase, nucleotide triphosphates (NTPs), and APS.

Preferably, the composition further comprises at least one selected from the group comprising a recombinase, NTPs and a single stranded nucleotide binding (SSB) protein. More preferably, the composition further comprises at least two selected from the group comprising a recombinase, NTPs and a single stranded nucleotide binding (SSB) protein.

Preferably, the composition may comprise a recombinase, NTPs and a single stranded nucleotide binding (SSB) protein.

Preferably, the composition may comprise ATP sulfurylase, a recombinase, a single-stranded nucleotide binding protein, a polymerase, nucleotide triphosphates (NTPs), and APS.

Preferably, the amplification composition comprises a recombinase. More preferably, the amplification composition further comprises a recombinase, a single-stranded nucleotide binding protein, a polymerase and a plurality of NTPs (e.g. dNTPs).

In some embodiments, the composition may not comprise one or more primers, either an amplification or a sequencing primer. Accordingly, the composition may not comprise primers. That is, the composition may not comprise any nucleic acid sequences that can initiate DNA synthesis (by a polymerase). The primers may be free nucleic acid sequence of between 18 and 22 base pairs, more preferably between 15 to 30 base pairs. The GC content of the free nucleic acid sequence may also be between 50 and 55%, and preferably, may have a GC-lock (a G or C in the last 5 bases of the sequence) at the 3′ end. The melting temperature of the primers may be between 40 and 60° C., more preferably between 50 and 55° C. The primers may also be complementary or substantially complementary (with e.g. at least 80% overall sequence identity) to a target sequence or complement thereof that the composition is intended to cluster. The primers may also comprise one or more restriction sites.

In some embodiment, the composition may also comprise a nucleic acid template. The nucleic acid template may also comprise the adaptor sequences described herein, where preferably the adaptor sequences comprise at least one of P5, P5′, P7 and P7′, the sequences of which are described below.

As used herein, the term “functional variant” refers to a variant polypeptide sequence or part of the polypeptide sequence which retains the biological function of the full non-variant sequence. For example, a functional variant of a functional variant of ATP sulfurylase is able to catalyze the conversion of PPi and adenylyl sulfate (APS) into ATP (and sulfate).

A functional variant also comprises a variant of the polypeptide of interest, which has sequence alterations that do not affect function, for example in non-conserved residues. Also encompassed is a variant that is substantially identical, i.e. has only some sequence variations, for example in non-conserved residues, compared to the wild type sequences as shown herein and is biologically active. Alterations in a polypeptide sequence that does not affect the functional properties of the polypeptide are well known in the art. For example, the amino acid alanine, a hydrophobic amino acid, may be substituted by another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Each of the proposed modifications is well within the routine skill in the art, as is determination of retention of biological activity of the encoded products.

As used in any aspect described herein, a “functional variant” has at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the non-variant amino acid sequence and preferably retains the catalytic activity of an ATP sulfurylase as described above. The sequence identity of a variant can be determined using any number of sequence alignment programs known in the art. As an example, Emboss Stretcher from the EMBL-EBI may be used: https://www.ebi.ac.uk/Tools/psa/emboss_stretcher/ (using default parameters: pair output format, Matrix=BLOSUM62, Gap open=1, Gap extend=1 for proteins; pair output format, Matrix=DNAfull, Gap open=16, Gap extend=4 for nucleotides).

As used herein, the term “functional fragment” refers to a functionally active series of consecutive amino acids from a longer polypeptide or protein. For example, a functional fragment may retain the catalytic activity of an ATP sulfurylase, as described above.

In one embodiment, the amplification composition may not comprise PEG.

In another embodiment, the amplification composition may also or alternatively not comprise luciferase and/or apyrase and/or luciferin.

The amplification composition may also or alternatively not comprise creatine kinase (CK) and/or creatine phosphate (CP).

The amplification composition may comprise a buffer. Preferably, the amplification composition is buffered to a pH of about 6.0 to about 9.0, preferably about 6.5 to about 8.8, more preferably about 7.5 to about 8.7, even more preferably about 8.3 to about 8.6.

The amplification composition may be supplied in a dry form (e.g. a freeze-dried form or a lyophilised form). In such a case, the amplification composition may be rehydrated, for example with water or a buffer solution, prior to use in amplification. In other embodiments, the amplification composition may be supplied as a solution (e.g. as an aqueous solution).

The composition may further comprise excipients. Suitable excipients may include surfactants, such as anionic surfactants, including alkyl sulfates (e.g. ammonium lauryl sulfate, sodium lauryl sulfate, sodium laureth sulfate, sodium myreth sulfate, sodium docusate), alkyl sulfonates (e.g. perfluorooctanesulfonate, perfluorobutanesulfonate), alkyl phosphates (e.g. alkyl-aryl ether phosphates, alkyl ether phosphates) and alkyl carboxylates (e.g. sodium stearate, sodium lauroyl sarcosinate, perfluorononanoate, perfluorooctanoate); cationic surfactants, including quaternary ammonium salts (e.g. cetrimonium bromide, cetylpyridinium chloride, benzalkonium chloride, benzethonium chloride, dimethyldioctadecylammonium chloride, dioctadecyldimethylammonium bromide); non-ionic surfactants, including fatty alcohol ethoxylates, alkylphenol ethoxylates, fatty acid ethoxylates, ethoxylated amines or fatty acid amides, poloxamers, polysorbates, (e.g. polyethylene glycol sorbitan alkyl esters (Tween)). Further excipients may include enzyme stabilisers, such as dithiothreitol (DTT), tris(2-carboxyethyl)phosphine (TCEP) and 2-mercaptoethanol (BME). Still further excipients may include molecular crowding agents such as polyethylene glycol (PEG), dextrans and epichlorohydrin-sucrose polymers (e.g. Ficoll); in some embodiments, PEG may be excluded.

In a further aspect, the present disclosure is directed to a kit comprising an amplification composition comprising ATP sulfurylase.

In some embodiments, the composition may not comprise one or more primers, either an amplification or a sequencing primer. Accordingly, the composition may not comprise primers. That is, the composition may not comprise any nucleic acid sequences that can initiate DNA synthesis (by a polymerase). The primers may be free nucleic acid sequence of between 18 and 22 base pairs, more preferably between 15 to 30 base pairs. The GC content of the free nucleic acid sequence may also be between 50 and 55%, and preferably, may have a GC-lock (a G or C in the last 5 bases of the sequence) at the 3′ end. The melting temperature of the primers may be between 40 and 60° C., more preferably between 50 and 55° C. The primers may also be complementary or substantially complementary (with e.g. at least 80% overall sequence identity) to a target sequence or complement thereof that the composition is intended to cluster. The primers may also comprise one or more restriction sites.

Preferably, the kit may comprise an amplification composition as described herein.

The kit may further comprise a recombinase as described herein. The recombinase may be provided separately from the (amplification) composition. For example, the recombinase may be in a different container to the (amplification) composition.

The kit may further comprise a single-stranded nucleotide binding protein as described herein. The single-stranded nucleotide binding protein may be provided separately from the (amplification) composition. For example, the single-stranded nucleotide binding protein may be in a different container to the (amplification) composition.

The kit may further comprise a polymerase as described herein. The polymerase may be provided separately from the (amplification) composition. For example, the polymerase may be in a different container to the (amplification) composition.

The kit may further comprise a plurality and mixture of nucleotide triphosphate (NTPs) as described herein. The nucleotide triphosphate may be provided separately from the (amplification) composition. For example, the nucleotide triphosphate may be in a different container to the (amplification) composition.

The kit may further comprise APS. The APS may be provided separately from the (amplification) composition. For example, the APS may be in a different container to the (amplification) composition.

The kit may further comprise another ATP-generating substrate as described herein (e.g. not including APS). The another ATP-generating substrate may be provided separately from the (amplification) composition. For example, the another ATP-generating substrate may be in a different container to the (amplification) composition. As described herein, the another ATP-generating substrate may be creatine phosphate (CP).

The kit may further comprise another ATP-generating enzyme as described herein (e.g. not including ATP sulfurylase). The another ATP-generating enzyme may be provided separately from the (amplification) composition. For example, the another ATP-generating enzyme may be in a different container to the (amplification) composition. As described herein, the another ATP-generating enzyme may be creatine kinase (CK).

The kit may comprise ATP sulfurylase, and at least one selected from the group consisting of: a recombinase, a single-stranded nucleotide binding protein, a polymerase, nucleotide triphosphates (NTPs), and APS. Preferably, the kit may comprise ATP sulfurylase, and at least two selected from the group consisting of: a recombinase, a single-stranded nucleotide binding protein, a polymerase, nucleotide triphosphates (NTPs), and APS. More preferably, the kit may comprise ATP sulfurylase, and at least three selected from the group consisting of: a recombinase, a single-stranded nucleotide binding protein, a polymerase, nucleotide triphosphates (NTPs), and APS. Even more preferably, the kit may comprise ATP sulfurylase, and at least four selected from the group consisting of: a recombinase, a single-stranded nucleotide binding protein, a polymerase, nucleotide triphosphates (NTPs), and APS. One or more (e.g. each of these components) may be provided separately from the (amplification) composition. For example, one or more (e.g. each of these components) may be in a different container to the (amplification) composition.

Preferably, the kit further comprises at least one selected from the group comprising a recombinase, NTPs and a single stranded nucleotide binding (SSB) protein. More preferably, the composition further comprises at least two selected from the group comprising a recombinase, NTPs and a single stranded nucleotide binding (SSB) protein. One or more (e.g. each of these components) may be provided separately from the (amplification) composition. For example, one or more (e.g. each of these components) may be in a different container to the (amplification) composition.

Preferably, the kit may comprise a recombinase, NTPs and a single stranded nucleotide binding (SSB) protein. One or more (e.g. each of these components) may be provided separately from the (amplification) composition. For example, one or more (e.g. each of these components) may be in a different container to the (amplification) composition.

Preferably, the kit may comprise ATP sulfurylase, a recombinase, a single-stranded nucleotide binding protein, a polymerase, nucleotide triphosphates (NTPs), and APS. One or more (e.g. each of these components) may be provided separately from the (amplification) composition. For example, one or more (e.g. each of these components) may be in a different container to the (amplification) composition.

The kit may further comprise excipients as described herein. The excipient(s) may be provided separately from the (amplification) composition. For example, the excipient(s) may be in a different container to the (amplification) composition.

The kit may further comprise one or more agents for use in preparing a template nucleic acid sequence for clustering and sequencing (i.e. library preparation agents). In one embodiment, the kit may further comprise adaptor sequences. The adaptor sequences may be configured such that they can be ligated onto a nucleic acid template to be sequenced. In some preferred embodiments, the kit may comprise a first adaptor sequence that comprises a sequence according to SEQ ID NO. 2 (P5) or a variant or fragment thereof In other preferred embodiments, the kit may comprise a second adaptor sequence that comprises a sequence according to SEQ ID NO. 3 (P7) or a variant or fragment thereof. In other preferred embodiments, the kit may comprise a third adaptor sequence that comprises a sequence according to SEQ ID NO. 4 (P5′) or a variant or fragment thereof. In other preferred embodiments, the kit may comprise a fourth adaptor sequence that comprises a sequence according to SEQ ID NO. 5 (P7′) or a variant or fragment thereof. More preferably, the kit may comprise at least two of the group selected from the first adaptor sequence, the second adaptor sequence, the third adaptor sequence and the fourth adaptor sequence. Even more preferably, the kit may comprise at least three of the group selected from the first adaptor sequence, the second adaptor sequence, the third adaptor sequence and the fourth adaptor sequence. Yet even more preferably, the kit may comprise the first adaptor sequence, the second adaptor sequence, the third adaptor sequence and the fourth adaptor sequence. The adaptor sequence(s) (e.g. each of the adaptor sequence(s)) may be provided separately from the (amplification) composition. For example, the adaptor sequence(s) (e.g. each of the adaptor sequence(s)) may be in a different container to the (amplification) composition.

The kit may further comprise a metal cofactor composition. The metal cofactor may be configured to activate one or more enzymes in the composition. For example, the metal cofactor may be configured to activate the recombinase and/or the polymerase. Preferably, the metal cofactor composition comprises magnesium ions (e.g. magnesium acetate, magnesium chloride). The metal cofactor composition may be provided separately from the (amplification) composition. For example, the metal cofactor composition may be in a different container to the (amplification) composition.

The kit may further comprise a solid support, preferably a flow cell. Preferably lawn primers (P5 and P7) are immobilised on the flow cell as described in detail above.

In a further aspect, the present disclosure is directed to use of an amplification composition as described herein, or a kit as described herein, in amplifying a nucleic acid template, or in sequencing a nucleic acid sequence.

In another aspect, there is provided a method of amplifying a nucleic acid template comprising reducing or removing inorganic pyrophosphate produced during the process of nucleic acid amplification. In another aspect, there is provided a method of increasing the amplification kinetics of a nucleic acid amplification reaction, the method comprising reducing or removing inorganic pyrophosphate produced during the process of nucleic acid amplification. The method may comprise adding the amplification composition to a sample containing a nucleic acid template to be amplified.

In another aspect, there is provided a method of resynthesis or improving resynthesis comprising reducing or removing inorganic pyrophosphate produced during clustering, by adding the composition during the resynthesis step. By re-synthesis is meant the step between the first and second sequencing reads where the template is copied using bridged strand resynthesis to produce a second immobilised template that is complementary to the first.

The method may comprise performing nucleic acid amplification at a temperature of about 50° C. to about 75° C., about 55° C. to about 70° C., or about 60° C. to about 65° C. This is called thermophilic clustering, and as described above, allows for increased clustering kinetics, decreased clustering times and faster end to end times for the user. Alternatively, amplification may be carried out isothermally.

In another aspect, there is provided a method of improving clustering or increasing the clustering kinetics, the method comprising removing or reducing the levels of inorganic pyrophosphate. Improving clustering may mean decreasing the time taken to form a cluster, as defined above and/or increasing the density/signal intensity of a cluster and/or increasing the integrity of the cluster/decreasing sequence-specific errors (i.e. faithful amplification of secondary structures within the genome, such as G-quadraplexes and the like). The improvement may be relative to clustering without the levels of pyrophosphate being reduced. An improvement or increase or decrease as used herein may be at least 20%, 30%, 40%, 50%, 60%, 70%, 80% or 90% or more.

The method may comprise adding the amplification composition as defined herein. The compositions may be added to a sample containing a nucleic acid template to be amplified. In particular, by “adding” may mean that the compositions are added to a flow cell before, after or at the same time as a sample containing the nucleic acid template. The nucleic acid template may contain the adaptor sequences (comprising at least one of P5, P5′, P7 and P7′) as described above.

The method may comprise adding the amplification composition only once. That is only one push of the amplification composition is required to generate a clonal cluster of sufficient density for later sequencing. Alternatively, the amplification composition may be added more than once—i.e. two or more times.

Amplification may be conducted by exclusion amplification. Amplification may be conducted by bridge amplification. Amplification may not be real-time PCR.

In a further embodiment, the present disclosure is directed to a method of sequencing a nucleic acid sequence, wherein the method comprises amplifying a nucleic acid template as described herein; and sequencing the amplified nucleic acid template.

The step of sequencing the amplified nucleic acid template may comprise performing a single read. In other embodiments, the step of sequencing the amplified nucleic acid template comprises performing a paired-end read.

The step of sequencing the amplified nucleic acid template may comprise conducting a first sequencing read and a second sequencing read.

The step of sequencing the amplified nucleic acid template may be conducted using a sequencing-by-synthesis technique or a sequencing-by-ligation technique. Preferably, the step of sequencing the amplified nucleic acid template pay be conducted using a sequencing-by-synthesis technique.

The method of sequencing a nucleic acid sequence may be conducted isothermally.

One or more steps in the method of sequencing a nucleic acid sequence are conducted at a temperature of about 50° C. to about 75° C., about 55° C. to about 70° C., or about 60° C. to about 65° C., for example, one or more steps may be conducted at about 50° C., about 55° C., about 60° C., about 65° C., about 70° C., or about 75° C. Preferably, all steps in the method of sequencing a nucleic acid sequence are conducted at a temperature of about 50° C. to about 75° C., about 55° C. to about 70° C., or about 60° C. to about 65° C., for example, all steps may be conducted at about 50° C., about 55° C., about 60° C., about 65° C., about 70° C., or about 75° C.

Where bridge amplification is used, the step of sequencing the amplified nucleic acid template may comprise a first linearisation step. The first linearisation step may be conducted after (e.g. immediately after) the step of amplifying a nucleic acid template.

The step of sequencing the amplified nucleic acid template may comprise a step of adding an exonuclease. The step of adding an exonuclease may be conducted after the step of amplifying a nucleic acid template. For example, the step of adding an exonuclease may be conducted after (e.g. immediately after) the first linearisation step.

Preferably, the exonuclease is a thermophilic exonuclease. More preferably, the exonuclease is derived from a thermophilic organism, such as Pyrococcus furious.

Preferably, the exonuclease has an optimum working temperature of about 50° C. to about 75° C., about 55° C. to about 70° C., or about 60° C. to about 65° C.

The step of sequencing the amplified nucleic acid template may comprise a first step of dehybridising (or denaturing) a complementary strand bound to the nucleic acid template with a dehybridisation/denaturation agent. The dehybridisation/denaturation agent may be configured to cause the complementary strand to detach from the nucleic acid template and thereby allow the complementary strand to be washed away. The first step of dehybridising a complementary strand may be conducted after the step of amplifying a nucleic acid template. For example, the first step of dehybridising a complementary strand may be conducted after (e.g. immediately after) the step of adding an exonuclease.

The step of sequencing the amplified nucleic acid template may comprise a first step of hybridising a sequencing primer onto the nucleic acid template. The first step of hybridising a sequencing primer may be conducted after the step of amplifying a nucleic acid template. For example, the first step of hybridising a sequencing primer may be conducted after (e.g. immediately after) the first step of dehybridising a complementary strand.

The step of sequencing the amplified nucleic acid template may comprise a first step of performing sequencing-by-synthesis. The first step of performing sequencing-by-synthesis may be conducted after the step of amplifying a nucleic acid template. For example, the first step of performing sequencing-by-synthesis may be conducted after (e.g. immediately after) the first step of hybridising a sequencing primer.

Where a second sequencing read (e.g. for a paired-end read) is conducted, the step of sequencing the amplified nucleic acid may further comprise a step of removing a blocking group from a hydroxyl group of a primer (e.g. a P5 or a P7 lawn primer). For example, the step of removing a blocking group may involve removal of a phosphate group using a blocking group phosphatase. The step of removing a blocking group may be conducted after the step of amplifying a nucleic acid template. For example, the step of removing a blocking group may be conducted after (e.g. immediately after) the first step of performing sequencing-by-synthesis.

Preferably, the blocking group phosphatase is a thermophilic phosphatase. More preferably, the blocking group phosphatase is derived from a thermophilic organism, such as Pyrococcus furious.

Preferably, the phosphatase has an optimum working temperature of about 50° C. to about 75° C., about 55° C. to about 70° C., or about 60° C. to about 65° C.

Where a second sequencing read (e.g. for a paired-end read) is conducted, the step of sequencing the amplified nucleic acid may further comprise a step of generating a complementary version of the amplified nucleic acid template. The step of generating a complementary version of the amplified nucleic acid template may involve using amplification methods as described herein. The step of generating a complementary version of the amplified nucleic acid template may be conducted after the step of amplifying a nucleic acid template. For example, the step of generating a complementary version of the amplified nucleic acid template may be conducted after (e.g. immediately after) the step of removing a blocking group.

Where a second sequencing read (e.g. for a paired-end read) is conducted, the step of sequencing the amplified nucleic acid template may comprise a second linearisation step. The second linearisation step may involve the use of an oxoguanine glycosylase (Ogg). The second linearisation step may be conducted after (e.g. immediately after) the step of generating a complementary version of the amplified nucleic acid template.

Preferably, the oxoguanine glycosylase is a thermophilic oxoguanine glycosylase. More preferably, the oxoguanine glycosylase is derived from a thermophilic organism, such as Methanococcus jannaschii.

Where a second sequencing read (e.g. for a paired-end read) is conducted, the step of sequencing the amplified nucleic acid template may comprise a second step of dehybridising a complementary strand bound to the (complementary version of the) nucleic acid template with a dehybridisation agent. The dehybridisation agent may be configured to cause the complementary strand to detach from the (complementary version of the) nucleic acid template and thereby allow the complementary strand to be washed away. The second step of dehybridising a complementary strand may be conducted after the step of amplifying a nucleic acid template. For example, the second step of dehybridising a complementary strand may be conducted after (e.g. immediately after) the second linearisation step.

Where a second sequencing read (e.g. for a paired-end read) is conducted, the step of sequencing the amplified nucleic acid template may comprise a second step of hybridising a sequencing primer onto the (complementary version of the) nucleic acid template. The second step of hybridising a sequencing primer may be conducted after the step of amplifying a nucleic acid template. For example, the second step of hybridising a sequencing primer may be conducted after (e.g. immediately after) the second step of dehybridising a complementary strand.

Where a second sequencing read (e.g. for a paired-end read) is conducted, the step of sequencing the amplified nucleic acid template may comprise a second step of performing sequencing-by-synthesis. The second step of performing sequencing-by-synthesis may be conducted after the step of amplifying a nucleic acid template. For example, the second step of performing sequencing-by-synthesis may be conducted after (e.g. immediately after) the second step of hybridising a sequencing primer.

The present disclosure will now be described by way of the following non-limiting examples.

### EXAMPLES

### Reference Example 1—Effect of Reducing PPi Using Pyrophosphatase (FIG. 5)

On board cluster generation (OBCG) was performed utilizing the NextSeq 2000 with a custom recipe to pull the ExAmp supplemented with 0.3 U PPiase per 100 μl clustering reagent or 1.2 U PPiase per 100 μl clustering reagent from a unique position within the sequencing cartridge. TruSeq Nano 450 (NA12878; source genomic DNA) supplemented with 1% PhiX v3 Control at a concentration of 300 pM was the seeded library. Two high output (HO) P3 flowcells and accompanying cartridges were utilized for each test condition. A single high output (HO) P3 flowcell was utilized as a control for comparison. A 2X151 sequencing run was executed for each of the flowcells. Primary metrics were pulled from sequence analysis viewer (SAV). The run was analyzed through the BaseSpace analysis workflow with DRAGEN Germline Alignment v3.7.5, downsample-bam, Firebrand R&D, which was automated with a wrapper in the AVATAR platform.

Overall, Reference Example 1 shows that improvements in various sequencing metrics can be obtained by reducing pyrophosphate levels.

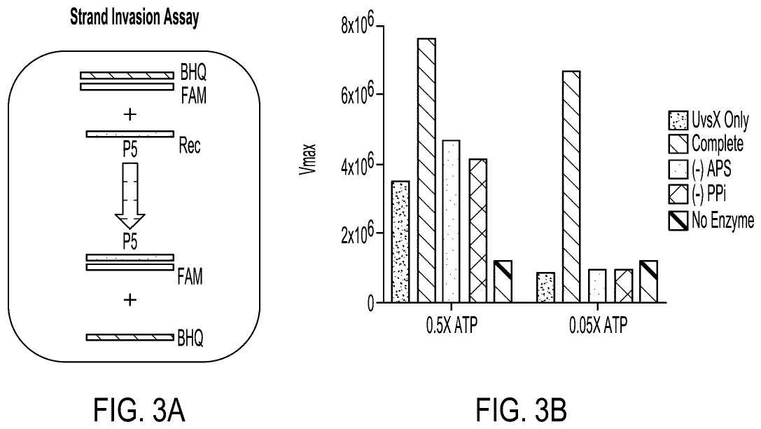

### Example 2—ATP Sulfurylase can Generate Energy to Power the Recombinase (FIG. 3)

An annealed duplex where one strand (red) has a black hole quencher (BHQ) attached and a strand (black) where a fluorescent dye (FAM) is attached. An oligonucleotide (oligo; green) with a region of homology to the annealed duplex (red) is present in the reaction tube. Recombinase, and combinations of the (+/−) of the energy regeneration system can be supplemented to the reaction tube. A fluorescent signal is generated when the BHQ strand is removed by the invading unlabeled strand allowing the FAM dye to be excited.

FIG. 3B shows that an ATP Sulfurylase can generate energy to power the recombinase.

### Example 3—ATP Sulfurylase Can Support RPA Reactions In Vitro (FIG. 4).

HCXE supported RPA reactions in the presence and absence of Sce ATP sulfurylase. M; DNA ladder with the sizes noted; lane 1 is the positive control with all components of HCXE; lane 2 HCXE with the energy system CK/CP removed, but 4.4 mM ATP present in the system; lane 3 HCXE with the energy system CK/CP removed, 4.4 mM ATP, 2 μg Sce ATP Sulfurylase and 4 mM APS; lane 4 negative control, no HCXE; just PhiX template; lane 5 HCXE with the energy system CK/CP removed, 4.4 mM ATP, and 4 mM APS.

### Additional Comments

While various illustrative examples are described above, it will be apparent to one skilled in the art that various changes and modifications may be made therein without departing from the disclosure. The appended claims are intended to cover all such changes and modifications that fall within the true spirit and scope of the embodiments described herein.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

SEQUENCE LISTING
SEQ ID NO: 2:
P5 sequence:
AATGATACGGCGACCACCGAGATCTACAC
SEQ ID NO: 3
P7 sequence:
CAAGCAGAAGACGGCATACGAGAT
SEQ ID NO: 4
P5′ sequence (complementary to P5):
GTGTAGATCTCGGTGGTCGCCGTATCATT
SEQ ID NO: 5
P7′ sequence (complementary to P7):
ATCTCGTATGCCGTCTTCTGCTTG

1. An amplification composition comprising ATP sulfurylase.

2. The amplification composition of claim 1, wherein the amplification composition comprises ATP sulfurylase at a concentration of about 0.01 pM to about 1000 μM.

3. The amplification composition of claim 1, wherein the composition further comprises adenylyl sulfate (APS).

4. The amplification composition of claim 3, wherein the amplification composition comprises APS at a concentration of about 0.01 μM to about 1000 μM.

5. The amplification composition of claim 1, wherein the composition further comprises at least one selected from the group comprising a polymerase, a recombinase, a plurality of nucleotide triphosphates (NTPs) and a single stranded nucleotide binding (SSB) protein.

6. (canceled)

7. The amplification composition of claim 1, wherein the ATP sulfurylase is derived from a mesophilic organism, wherein the mesophilic organism comprises Saccharomyces cerevisiae or E. coli.

8. The amplification composition of claim 1, wherein the ATP sulfurylase comprises an amino acid sequence as defined in SEQ ID NO: 1 or a functional variant or fragment thereof.

9. The amplification composition of claim 5, wherein the polymerase is DNA Polymerase I and the recombinase is Recombinase A.

10. The amplification composition of claim 1, wherein the composition does not comprise PEG.

11. The amplification composition claim 1, wherein the amplification composition comprises a buffer, and wherein the composition is buffered to a pH of about 6.0 to about 9.0.

12. The amplification composition of claim 1, wherein the amplification composition is a clustering composition or a sequencing-by-synthesis composition or a resynthesis composition.

13. A kit comprising the amplification composition according to claim 1.

14. The kit of claim 13, wherein the kit further comprises a metal cofactor composition, wherein the metal cofactor composition comprises magnesium ions.

15. Use of the amplification composition of claim 1 to amplify a nucleic acid sequence.

16. Use of the amplification composition of claim 1 to form a monoclonal cluster of a template nucleic acid sequence.

17. A method of amplifying a target nucleic acid template, the method comprising adding the amplification composition of claim 1 to a sample comprising the nucleic acid template.

18. A method of forming a monoclonal cluster of a template nucleic acid sequence, the method comprising adding the amplification composition of claim 1 to a sample comprising the nucleic acid template.

19. The method of claim 17, wherein nucleic acid amplification is performed at a temperature of between 20 and 45° C.

20. The method of claim 17, wherein the method comprises adding the amplification composition only once.

21. A method of sequencing a nucleic acid sequence, wherein the method comprises:
amplifying a nucleic acid template using the method of claim 17; and
sequencing the amplified nucleic acid template.

22. A method according to claim 21, wherein the step of sequencing the amplified nucleic acid template comprises conducting a first sequencing read and a second sequencing read.

23. A method according to claim 21, wherein the step of sequencing the amplified nucleic acid template is conducted using a sequencing-by-synthesis technique or a sequencing-by-ligation technique.
